(19) United States
(12) Patent Application Publication
(10) Pub. No.: US 2025/0204912 A1
(43) Pub. Date: Jun. 26, 2025

(54) SURGICAL STAPLE CARTRIDGE WITH AN IMPROVED STAPLE DRIVER RETAINING FEATURE

(71) Applicant: Cilag GmbH International, Zug (CH)

(72) Inventors: Mengli Yang, Shanghai (CN); Anil K. Nalagatla, Cincinnati, OH (US)

(21) Appl. No.: 18/851,271

(22) PCT Filed: Apr. 5, 2023
(86) PCT No.: PCT/IB2023/053467

§ 371 Date: Sep. 26, 2024

(30) Foreign Application Priority Data
Apr. 8, 2022 (CN) 202210369850.3

(51) Int. Cl. A61B 17/072; A61B 17/00

(52) U.S. Cl. CPC A61B 17/07207; A61B 2017/00477; A61B 2017/07271

(57) ABSTRACT

The present disclosure provides a surgical staple cartridge. The surgical staple cartridge comprises two opposite side walls extending along a longitudinal direction which is perpendicular to a staple driving direction, and at least one side wall is provided with one or more cantilever beams along the longitudinal direction, the cantilever beams extend parallel to the staple driving direction, wherein the cantilever beams are provided with protrusions or grooves which are configured to cooperate with grooves or protrusions formed on a staple driver so as to hold the staple driver in place, and the cantilever beams are configured to deflect outwards to allow the passing through of the staple driver.

### FIELD

The present disclosure relates to the field of surgical instruments, in particular, to the field of surgical stapler/anastomat.

### BACKGROUND

A surgical stapler/anastomat is usually used to deploy staples into tissue for reducing or eliminating tissue bleeding, for example, it is necessary to seal the tissue as the tissue being cut in order to promote healing. A surgical stapler/anastomat (such as a linear stapler/anastomat, a right-angle stapler/anastomat) may comprise an end effector assembly having a staple cartridge portion and a staple anvil portion, wherein the staple cartridge portion comprises a staple cartridge configured to removably store surgical staples therein, and the staple anvil portion comprises staple forming pockets for shaping staples. Such surgical stapler/anastomat generally comprises a closure system that moves one of the staple cartridge and the staple anvil relative to the other and a staple driver for driving the staples.

As described above, the surgical stapler/anastomat may be configured to close the staple cartridge portion and the staple anvil portion of the end effector assembly into a closed configuration, so as to capture tissue between the staple cartridge portion and the staple anvil portion. Then, the staple driver pushes the staples to deploy from the staple cartridge and form against the staple forming grooves of the staple anvil portion through the tissue, thereby fixing tissue layers together. The staples are generally deployed in several staple lines to fix the tissue layers together more reliably. The end effector assembly may comprise or not comprise a cutting member which can be advanced between the staple lines, so as to cut the tissue after the tissue layers have been sealed together.

The staple cartridge comprises a staple driver retaining feature in order to minimize or prevent accidental staple push out during such as transportation. The staple driver retaining feature is generally elastic or deformable. In case that the staples are needed to be fired, a force will be applied to the staple driver to push the staple driver to advance forward. Under an appropriate force, the staple driver retaining feature will deflect or bend outwards so as to release the staple driver, allowing the staple driver to advance forward and in turn push the staple from the staple cartridge.

In the existing surgical staple cartridges, the staple driver retaining feature is generally in the form of clamping walls, which are usually side walls of the staple cartridge. The staple driver is clamped between the side walls. The side walls will deflect or bend outwards so as to release the staple driver when a sufficient push force is applied to the staple driver to push the staple driver to advance forward. The structure and size of the side walls are associated with the entire staple cartridge or other parts of the staple cartridge, it is difficult to predict or control a retaining force of the staple driver retaining feature.

Therefore, there is a need for an improved surgical staple cartridge to eliminate or reduce the foregoing defects.

### SUMMARY OF THE DISCLOSURE

The present disclosure provides a surgical staple cartridge with improved staple driver retaining features which isolate the hold and release mechanism from rest of the side walls of the staple cartridge so as to provide an appropriate retaining force for the staple driver.

The present disclosure provides a surgical staple cartridge comprising: a first side wall extending along a longitudinal direction which is perpendicular to a staple driving direction, the first side wall is provided with one or more first cantilever beams along the longitudinal direction, the first cantilever beams extend parallel to the staple driving direction, each first cantilever beam is provided with a protrusion or groove, a second side wall arranged opposite to and parallel to the first side wall, the protrusion or groove formed on the first cantilever beam is configured to cooperate with a groove or protrusion formed on a first side of the staple driver to hold the staple driver in place, and the first cantilever beam is configured to deflect outwards so as to allow the staple driver to advance forward.

According to a preferred embodiment, the first cantilever beams are equidistantly spaced along the longitudinal direction.

According to a preferred embodiment, the first cantilever beams are not equidistantly spaced along the longitudinal direction.

According to a preferred embodiment, an outer surface of the first cantilever beam is inwardly or outwardly inclined at an angle relative to an outer surface of the first side wall.

According to a preferred embodiment, an outer surface of the first cantilever beam and an outer surface of the first side wall are in the same plane or parallel to each other.

According to a preferred embodiment, an inner surface of the first cantilever beam is inwardly or outwardly inclined at an angle relative to an inner surface of the first side wall.

According to a preferred embodiment, wherein an inner surface of the first cantilever beam and an inner surface of the first side wall are in the same plane or parallel to each other.

According to a preferred embodiment, the second side wall is provided with one or more second cantilever beams along the longitudinal direction, the cantilever beams extend parallel to the staple driving direction, each second cantilever beam is provided with a protrusion or groove, the protrusion or groove formed on the second cantilever beam is configured to cooperate with a groove or protrusion formed on a second side of the staple driver opposite to the first side so as to hold the staple driver in place, wherein the second cantilever beam is configured to deflect outwards to allow the staple driver to advance forward.

According to a preferred embodiment, the second cantilever beams are equidistantly spaced along the longitudinal direction.

According to a preferred embodiment, the second cantilever beams are not equidistantly spaced along the longitudinal direction.

According to a preferred embodiment, the one or more first cantilever beams are respectively aligned with the one or more second cantilever beams along the longitudinal direction.

According to a preferred embodiment, the one or more first cantilever beams are staggered with the one or more second cantilever beams along the longitudinal direction.

According to a preferred embodiment, an outer surface of the second cantilever beam is inwardly or outwardly inclined at an angle relative to an outer surface of the second side wall.

According to a preferred embodiment, an outer surface of the second cantilever beam and an outer surface of the second side wall are in the same plane or parallel to each other.

According to a preferred embodiment, an inner surface of the second cantilever beam is inwardly or outwardly inclined at an angle relative to an inner surface of the second side wall.

According to a preferred embodiment, an inner surface of the second cantilever beam and an inner surface of the second side wall are in the same plane or parallel to each other.

According to a preferred embodiment, the protrusion formed on the cantilever beam or the staple driver is a bar extending along the longitudinal direction.

According to a preferred embodiment, a cross section of the protrusion formed on the cantilever beam or the staple driver is trapezoidal or cambered.

The surgical staple cartridge provided by the present disclosure has staple driver retaining features that can deflect or bend independently from the side walls of the staple cartridge, such as cantilever retaining features. The cantilever retaining features can be designed to some certain extent independently from the side walls of the staple cartridge, and can deflect or deform independently from the side walls of the staple cartridge under a pushing force of the staple driver so as to allow the staple driver to be released, such that a desired retaining force is provided for the staple driver without changing an entire structure of the side walls of the staple cartridge.

### BRIEF DESCRIPTION OF THE DRAWINGS

The present disclosure and its features and advantages will be better understood with reference to the following description of exemplary embodiments of the present disclosure in conjunction with the accompanying drawings. In the following description and drawings, similar components are indicated with similar reference signs. The figures are not necessarily drawn to scale, and for the sake of clarity and conciseness, some parts may be omitted, and some figures may be drawn in an exaggerated or sketchy way.

FIG. 1 exemplarily shows a surgical stapler without a surgical staple cartridge installed therein;

FIG. 2 exemplarily shows an end effector portion of the surgical stapler with a surgical staple cartridge installed therein;

FIG. 3 exemplarily shows a side view of the surgical staple cartridge according to the present disclosure;

FIG. 4 exemplarily shows a perspective view of the surgical staple cartridge according to the present disclosure;

FIG. 5 exemplarily shows a longitudinal cross-sectional view of the surgical staple cartridge according to the present disclosure;

FIG. 6 exemplarily shows a partial cross-sectional view of the surgical staple cartridge according to the present disclosure.

### DETAILED DESCRIPTION OF EMBODIMENTS

Next, exemplary embodiments of the present disclosure will be described in detail with reference to the accompanying drawings. Described herein are merely exemplary embodiments in accordance with the present disclosure, and those skilled in the art will envisage other ways to implement the present disclosure on the basis of the exemplary embodiments described herein, which also fall within the scope of the present disclosure.

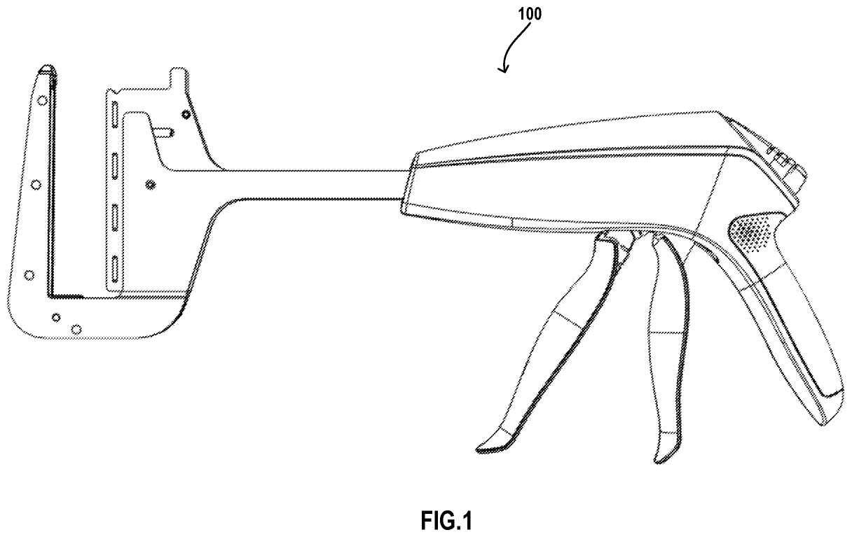

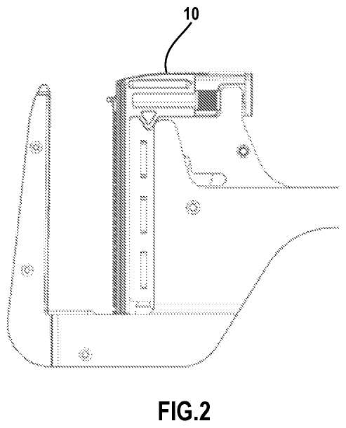

FIG. 1 illustrates a surgical stapler 100. A surgical staple cartridge 10 according to the present disclosure may be installed in the surgical stapler 100 and other similar surgical staplers. FIG. 2 illustrates an end effector portion of the surgical stapler 100 installed with the surgical staple cartridge 10.

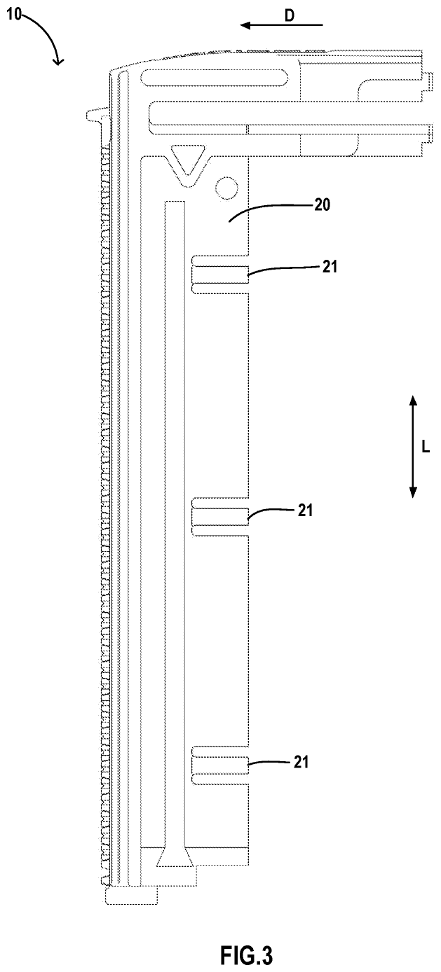

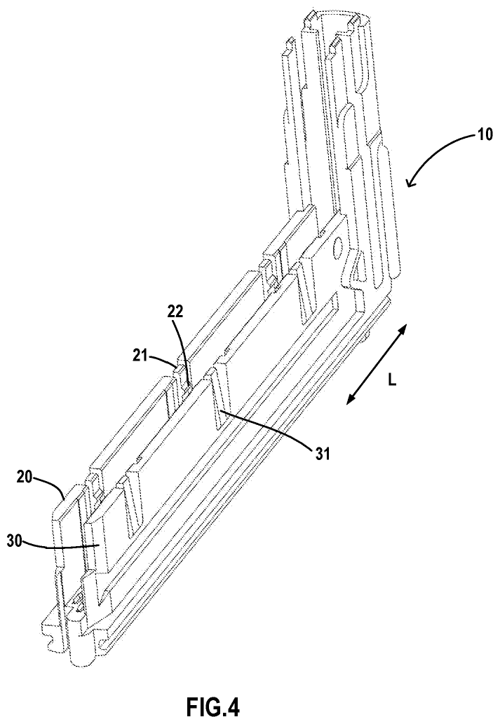

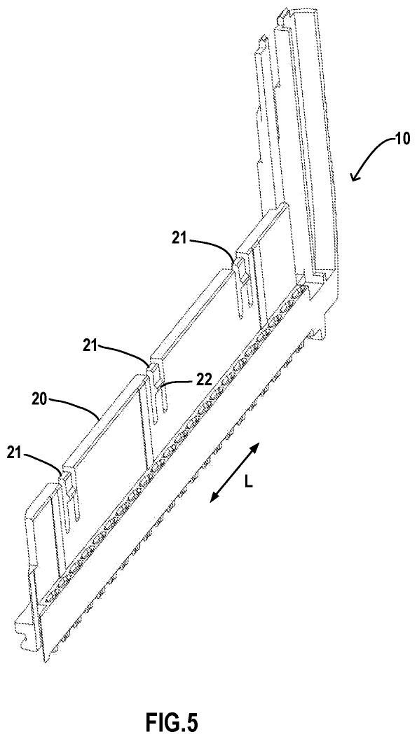

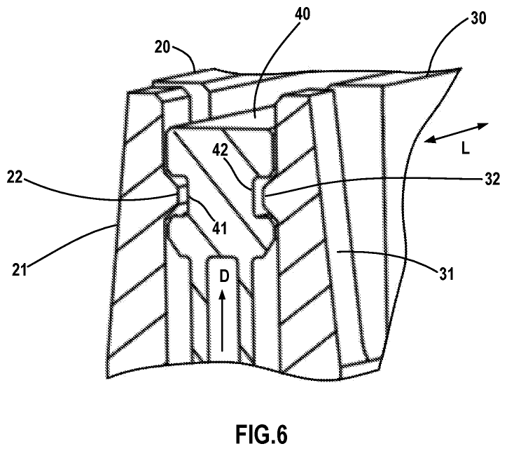

FIG. 3 and FIG. 4 illustrate a surgical staple cartridge 10 according to the present disclosure, and FIG. 5 and FIG. 6 illustrate a longitudinal sectional view and a traverse sectional view of the staple cartridge 10, respectively. As shown in the figures, the surgical staple cartridge 10 comprises side walls 20 and 30 extending along a longitudinal direction L, and the side walls 20 and 30 are arranged parallel and opposite to each other.

In order to hold the staple driver 40 (seen in FIG. 6) in place properly, one or more cantilever beams 21, 31 are provided in the side walls 20, 30 respectively. The cantilever beams may be integrally formed with the side walls. The ends of the cantilever beams are substantially flush with the end of the side walls. There are slots provided at the upper side and the lower side of each cantilever beam, which form the individual cantilever beam from the side wall. Each cantilever beam is respectively provided with a protrusion 22, 32 (preferably on an inner surface thereof). The protrusion 22, 32 can cooperate with grooves 41, 42 on both sides of the staple driver 40 extending along the longitudinal direction L, so as to hold the staple driver 40 in place and prevent the staple driver 40 from moving along a driving direction D by accident which may lead to accidental staple push out.

When an appropriate force is applied to the staple driver 40 to advance the staple driver toward the staples along the direction D, the side walls 20, 30 will be deflected outwards by the staple driver 40 so as to release the staple driver, allowing the staple driver to push the staples out of the staple cartridge. The cantilever beams 21, 31 and the protrusions 22, 32 can be designed to apply an appropriate retaining force to the staple driver 40 without revising rest of the side walls 20, 30.

It will be understood that, in the staple cartridge of the present disclosure, it is not necessary to provide the cantilever beams in both side walls 20, 30 Instead, the purpose of the present disclosure may also be achieved by providing the cantilever structure in only one side wall. It will also be understood that, alternatively or additionally, the cantilever beams may be provided with grooves while the staple driver may be provided with protrusions which are configured to cooperate with the grooves formed on the cantilever beams.

Similarly, in the staple cartridge of the present disclosure, each side wall 20, 30 may be provided with a same or different number of the cantilever structures, and the cantilever structures may be evenly arranged along the longitudinal direction L on each side wall 20, 30, or unevenly arranged. Furthermore, the cantilever structures on the side wall 20 may be aligned or staggered with the cantilever structures on the side wall 30 along the longitudinal direction.

Furthermore, as shown in FIG. 6, in a preferred embodiment of the present disclosure, outer surfaces of the cantilever beams 21 and/or the cantilever beams 31 may be inwardly inclined at a certain angle relative to outer surfaces of the corresponding side walls 20, 30, while the inner surfaces of the cantilever beams and inner surfaces of the corresponding side walls are positioned in the same plane, that is, the cross-section of the cantilever beam is tapered. The cantilever beams with such configuration possess better deformability or is easier to deflect outwards, such that only a small force is required to overcome the retaining force on the staple driver 40 applied by the cantilever structures so as to release the staple driver. Therefore, in the staple cartridge of the present disclosure, the shape of each cantilever beam may be designed independently from rest of the side walls, and the inner and outer surfaces of the cantilever beam may be inwardly or outwardly inclined at an angle relative to the inner and outer surfaces of the side walls. In other embodiments, the inner and outer surfaces of the cantilever beam may be respectively aligned with the inner and outer surfaces of the side walls (positioned in a same plane). In further embodiments, the inner and outer surfaces of the cantilever beam may be parallel to the inner and outer surfaces of the side walls (positioned in different planes). The specific structure of the cantilever beam may be designed according to a desired retaining force, including extending directions of the inner and outer surfaces of the cantilever beams, shapes and sizes of the protrusions (or grooves), etc. In a further embodiment, the inner and outer surfaces of the cantilever beams may be curved surfaces.

In a preferred embodiment of the present disclosure, the protrusions 22, 32 are bars extending along the longitudinal direction L (as shown in FIG. 4 and FIG. 5), and the cross sections of the protrusions 22, 32 are trapezoidal (as shown in FIG. 6). It is conceivable that the cross sections of the protrusions 22, 32 may have other shapes such as cambered shape, as long as they can provide the desired retaining force for holding the staple driver 40. Similarly, the protrusions formed on the staple driver may be bars extending along the longitudinal direction L, of which the cross sections may be trapezoidal, cambered or of any other shapes.

The scope of protection of the present disclosure is defined only by the claims. References in this specification to “various embodiments”, “some embodiments”, “one embodiment” or “an embodiment” mean that specific features, structures or characteristics described in conjunction with the embodiments are included in at least one embodiment. Therefore, the phrases “in various embodiments”, “in some embodiments”, “in one embodiment” or “in an embodiment” and the like in the present specification are not necessarily all referring to the same embodiment. Furthermore, in one or more embodiments, the specific features, structures or characteristics may be combined in any suitable way. Therefore, without conflict, the specific features, structures or characteristics shown or described in conjunction with one embodiment can be fully or partially combined with the features, structures or characteristics of one or more other embodiments, and the resulting modifications and variations are also within the scope of the present disclosure.

1. A surgical staple cartridge for a surgical stapler, wherein a staple driver of the surgical stapler is operable to push staples out of the surgical staple cartridge, the surgical staple cartridge comprising:
a first side wall extending along a longitudinal direction which is perpendicular to a staple driving direction, the first side wall including one or more first cantilever beams arranged along the longitudinal direction, the one or more first cantilever beams extending parallel to the staple driving direction, and each first cantilever beam including a protrusion or groove; and
a second side wall arranged opposite to and parallel to the first side wall;
wherein the protrusion or groove formed on the first cantilever beam is configured to cooperate with a groove or protrusion formed on a first side of the staple driver to hold the staple driver in place, and wherein the first cantilever beam is configured to deflect outwards to thereby allow the staple driver to pass through the staple cartridge.

2. The surgical staple cartridge according to claim 1, wherein the first cantilever beams are equidistantly spaced along the longitudinal direction.

3. The surgical staple cartridge according to claim 1, wherein the first cantilever beams are not equidistantly spaced along the longitudinal direction.

4. The surgical staple cartridge according to claim 1, wherein an outer surface of the first cantilever beam is inwardly or outwardly inclined at an angle relative to an outer surface of the first side wall.

5. The surgical staple cartridge according to claim 1, wherein an outer surface of the first cantilever beam and an outer surface of the first side wall are in the same plane or parallel to each other.

6. The surgical staple cartridge according to claim 1, wherein an inner surface of the first cantilever beam is inwardly or outwardly inclined at an angle relative to an inner surface of the first side wall.

7. The surgical staple cartridge according to claim 1, wherein an inner surface of the first cantilever beam and an inner surface of the first side wall are in the same plane or parallel to each other.

8. The surgical staple cartridge according to claim 1, wherein the second side wall includes one or more second cantilever beams arranged along the longitudinal direction, the one or more second cantilever beams extending parallel to the staple driving direction, each second cantilever beam being provided with a protrusion or groove, wherein the protrusion or groove formed on the second cantilever beam is configured to cooperate with a groove or protrusion formed on a second side opposite to the first side of the staple driver so as to hold the staple driver in place, and wherein the second cantilever beam is configured to deflect outwards to thereby allow the staple driver to pass through the staple cartridge.

9. The surgical staple cartridge according to claim 8, wherein the second cantilever beams are equidistantly spaced along the longitudinal direction.

10. The surgical staple cartridge according to claim 8, wherein the second cantilever beams are not equidistantly spaced along the longitudinal direction.

11. The surgical staple cartridge according to claim 8, wherein the one or more first cantilever beams are respectively aligned with the one or more second cantilever beams along the longitudinal direction.

12. The surgical staple cartridge according to claim 8, wherein the one or more first cantilever beams are staggered with the one or more second cantilever beams along the longitudinal direction.

13. The surgical staple cartridge according to claim 8, wherein an outer surface of the second cantilever beam is inwardly or outwardly inclined at an angle relative to an outer surface of the second side wall.

14. The surgical staple cartridge according to claim 8, wherein an outer surface of the second cantilever beam and an outer surface of the second side wall are in the same plane or parallel to each other.

15. The surgical staple cartridge according to claim 8, wherein an inner surface of the second cantilever beam is inwardly or outwardly inclined at an angle relative to an inner surface of the second side wall.

16. The surgical staple cartridge according to claim 8, wherein an inner surface of the second cantilever beam and an inner surface of the second side wall are in the same plane or parallel to each other.

17. The surgical staple cartridge according to claim 8, wherein the protrusion comprises a bar extending along the longitudinal direction.

18. The surgical staple cartridge according to claim 8, wherein a cross section of the protrusion formed on the second cantilever beam or the second side of the staple driver is trapezoidal or cambered.

19. The surgical staple cartridge according to claim 1, wherein a cross section of the protrusion formed on the first cantilever beam or the first side of the staple driver is trapezoidal or cambered.
